(19) United States
(12) Patent Application Publication
(10) Pub. No.: US 2022/0087730 A1
(43) Pub. Date: Mar. 24, 2022

(54) THERMAL CUTTING-ASSISTED TISSUE RESECTION DEVICES AND SYSTEMS

(71) Applicant: Covidien LP, Mansfield, MA (US)

(72) Inventors: Richard L. Croft, Mead, CO (US); Matthew S. Cowley, Frederick, CO (US)

(21) Appl. No.: 17/465,202

(22) Filed: Sep. 2, 2021

Related U.S. Application Data
(60) Provisional application No. 63/081,387, filed on Sep. 22, 2020.

(51) Int. Cl. A61B 18/08

(52) U.S. Cl. CPC A61B 18/082; A61B 17/32053

(57) ABSTRACT

A tissue resection device includes a handpiece and an end effector assembly. The end effector assembly includes an outer shaft defining a window, an inner shaft rotationally disposed within the outer shaft and operably coupled to the handpiece for rotating the inner shaft relative to the outer shaft, a cutting element disposed at a distal end portion of the inner shaft within the outer shaft and rotatable with the inner shaft, and at least one electromagnetic induction coil surrounding at least a portion of the cutting element. The cutting element is formed at least partially from a ferromagnetic material capable of being inductively heated and the at least one electromagnetic induction coil is adapted to connect to a source of energy to produce an electromagnetic field within the at least one electromagnetic induction coil to thereby inductively heat the cutting element.

### CROSS-REFERENCE TO RELATED APPLICATIONS

This application claims the benefit of, and priority to, U.S. Provisional Patent Application No. 63/081,387 filed on Sep. 22, 2020. The entire contents of each of these applications is hereby incorporated herein by reference.

### BACKGROUND

### Technical Field

The present disclosure relates generally to surgical devices and systems and, more particularly, to thermal cutting-assisted tissue resection devices and systems.

### Background of Related Art

Tissue resection may be performed endoscopically by inserting an endoscope into an internal surgical site and passing a tissue resection device through the endoscope and into the internal surgical site. With respect to such endoscopic tissue resection procedures, it often is desirable to distend the internal surgical site with a fluid, for example, saline, sorbitol, or glycine. The inflow and outflow of the fluid during the procedure maintains the internal surgical site in a distended state and flushes tissue and other debris therefrom to maintain a visible working space. Tissue resection may also be performed in open and/or other surgical procedures.

### SUMMARY

As used herein, the term “distal” refers to the portion that is being described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with the present disclosure is a tissue resection device including a handpiece and an end effector assembly extending distally from the handpiece. The end effector assembly includes an outer shaft including a window defined therethrough towards a distal end thereof, an inner shaft rotationally disposed within the outer shaft, a cutting element disposed at a distal end portion of the inner shaft within the outer shaft and at least partially overlapping the window, and at least one electromagnetic induction coil surrounding at least a portion of the cutting element. The inner shaft is operably coupled to the handpiece such that the handpiece is capable of rotationally driving the inner shaft relative to the outer shaft. The cutting element is configured such that rotation of the inner shaft rotates the cutting element. The cutting element is formed at least partially from a ferromagnetic material capable of being inductively heated. The at least one electromagnetic induction coil is adapted to connect to a source of energy to produce an electromagnetic field within the at least one electromagnetic induction coil to thereby inductively heat the cutting element.

In an aspect of the present disclosure, the cutting element defines at least one cutting edge configured to cut tissue upon at least one of heating of the cutting element or rotation of the cutting element.

In another aspect of the present disclosure, the cutting element defines a screw-shaped configuration including at least one helical cutting edge.

In still another aspect of the present disclosure, the cutting element includes an elongated body and a plurality of elongated arms annularly spaced about the elongated body. Each elongated arm of the plurality of elongated arms defines at least one elongated cutting edge. In such aspects, each elongated arm of the plurality of elongated arms may define an elongated cutting edge along each side thereof.

In yet another aspect of the present disclosure, the at least one electromagnetic induction coil includes an electromagnetic induction coil disposed on or embedded within the outer shaft. Alternatively, the at least one electromagnetic induction coil may include a plurality of electromagnetic induction coils disposed about different portions of the cutting element, e.g., arms extending from a body of the cutting element.

In still yet another aspect of the present disclosure, the handpiece includes a motor and an output coupler connected to the motor and the end effector assembly includes an input coupler connected to the inner shaft. The input coupler is configured to engage the output coupler to enable the motor to drive rotation of the inner shaft.

In another aspect of the present disclosure, the end effector assembly is configured to releasably engage the handpiece to thereby releasably couple the output and input couplers with one another.

In yet another aspect of the present disclosure, the handpiece is adapted to connect to a vacuum source to establish suction through the outer shaft, thereby facilitating drawing tissue through the window and into the outer shaft for cutting with the cutting element.

In still another aspect of the present disclosure, the cutting element is formed from a ferromagnetic material.

Another tissue resection device provided in accordance with the present disclosure includes a handpiece and an end effector assembly extending distally from the handpiece. The end effector assembly includes an outer shaft including a window defined therethrough towards a distal end thereof, an electromagnetic induction coil disposed on or embedded within the outer shaft, an inner shaft rotationally disposed within the outer shaft and operably coupled to the handpiece such that the handpiece is capable of rotationally driving the inner shaft relative to the outer shaft, and a cutting element disposed at a distal end portion of the inner shaft within the outer shaft and at least partially overlapping the window and the electromagnetic induction coil. The cutting element is configured such that rotation of the inner shaft rotates the cutting element. The electromagnetic induction coil is adapted to connect to a source of energy to produce an electromagnetic field within the outer shaft. The cutting element is formed at least partially from a ferromagnetic material capable of being inductively heated by the electromagnetic field produced by the electromagnetic induction coil.

In an aspect of the present disclosure, the cutting element defines a screw-shaped configuration including at least one helical cutting edge.

In another aspect of the present disclosure, the handpiece includes a motor and an output coupler connected to the motor, the end effector assembly includes an input coupler connected to the inner shaft, and the input coupler is configured to engage the output coupler to enable the motor to drive rotation of the inner shaft.

In another aspect of the present disclosure, the handpiece is adapted to connect to a vacuum source to establish suction through the outer shaft, thereby facilitating drawing tissue through the window and into the outer shaft for cutting with the cutting element.

Still another tissue resection device provided in accordance with the present disclosure includes a handpiece and an end effector assembly extending distally from the handpiece. The end effector assembly includes an outer shaft including a window defined therethrough towards a distal end thereof, an inner shaft rotationally disposed within the outer shaft and operably coupled to the handpiece such that the handpiece is capable of rotationally driving the inner shaft relative to the outer shaft, a cutting element disposed at a distal end portion of the inner shaft within the outer shaft and at least partially overlapping the window, and a plurality of electromagnetic induction coils. The cutting element is configured such that rotation of the inner shaft rotates the cutting element. The cutting element is formed at least partially from a ferromagnetic material capable of being inductively heated and includes a body having a plurality of arms disposed annularly thereabout. Each electromagnetic induction coil of the plurality of electromagnetic induction coils is disposed about an arm of the plurality of arms. Each electromagnetic induction coil is adapted to connect to a source of energy to produce an electromagnetic field therewithin to thereby inductively heat the corresponding arm of the cutting element.

In an aspect of the present disclosure, each arm of the plurality of arms defines an elongated cutting edge along each side thereof.

In another aspect of the present disclosure, the handpiece includes a motor and an output coupler connected to the motor, the end effector assembly includes an input coupler connected to the inner shaft, and the input coupler is configured to engage the output coupler to enable the motor to drive rotation of the inner shaft.

In another aspect of the present disclosure, the handpiece is adapted to connect to a vacuum source to establish suction through the outer shaft, thereby facilitating drawing tissue through the window and into the outer shaft for cutting with the cutting element.

### BRIEF DESCRIPTION OF THE DRAWINGS

Various aspects and features of the present disclosure are described hereinbelow with reference to the drawings wherein like numerals designate identical or corresponding elements in each of the several views.

FIG. 1 is a perspective view of a tissue resecting system in accordance with the present disclosure configured for use in hysteroscopic and/or other surgical procedures;

FIG. 2A is an enlarged, perspective view of a distal end portion of an end effector assembly of a tissue resecting device of the tissue resecting system of FIG. 1;

FIG. 2B is an enlarged, perspective, partial cut-away view of the distal end portion of the end effector assembly of FIG. 2A;

FIG. 3A is a longitudinal, cross-sectional view of a portion of the tissue resecting device of the tissue resecting system of FIG. 1, wherein the end effector assembly is disengaged from the handpiece;

FIG. 3B is a longitudinal, cross-sectional view of the portion of the tissue resecting device illustrated in FIG. 4A, wherein the end effector assembly is engaged with the handpiece;

FIG. 4A is an enlarged, perspective view of a distal end portion of an inner shaft of the tissue resecting device of FIG. 1 including another cutting element in accordance with the present disclosure engaged thereon;

FIG. 4B is a distal end view of the cutting element of FIG. 4A; and

FIG. 5 is a schematic illustration of a robotic surgical system in accordance with the present disclosure.

### DETAILED DESCRIPTION

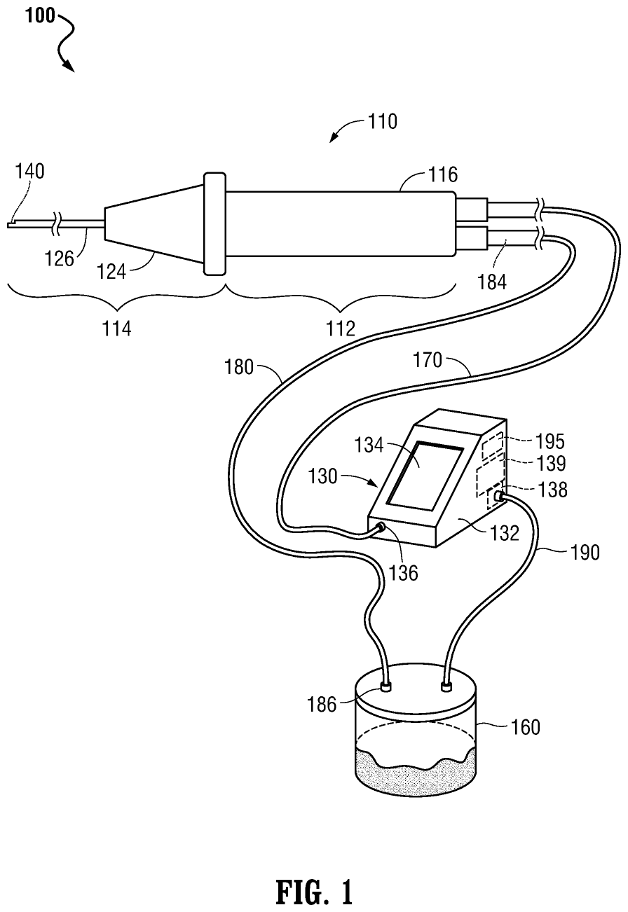

Referring to FIG. 1, a surgical system provided in accordance with aspects of the present disclosure is shown generally identified by reference numeral 100. Surgical system 100 includes a surgical device 110, a control console 130, and a collection vessel 160. Surgical system 100 further includes a cable 170, outflow tubing 180, and vacuum tubing 190. Surgical system 100 may further include an endoscope (not shown), e.g., a hysteroscope, defining a working channel for inserting of surgical device 110 therethrough, and adapted to connect to inflow tubing (not shown) to supply fluid to an internal surgical site and/or additional outflow tubing (not shown) to return fluid to collection vessel 160 (or another collection vessel). Alternatively, surgical system 100 may be used in other surgical procedures, e.g., itself or inserted through a laparoscope (not shown), in open surgical procedures, in robotic surgical procedures, etc.

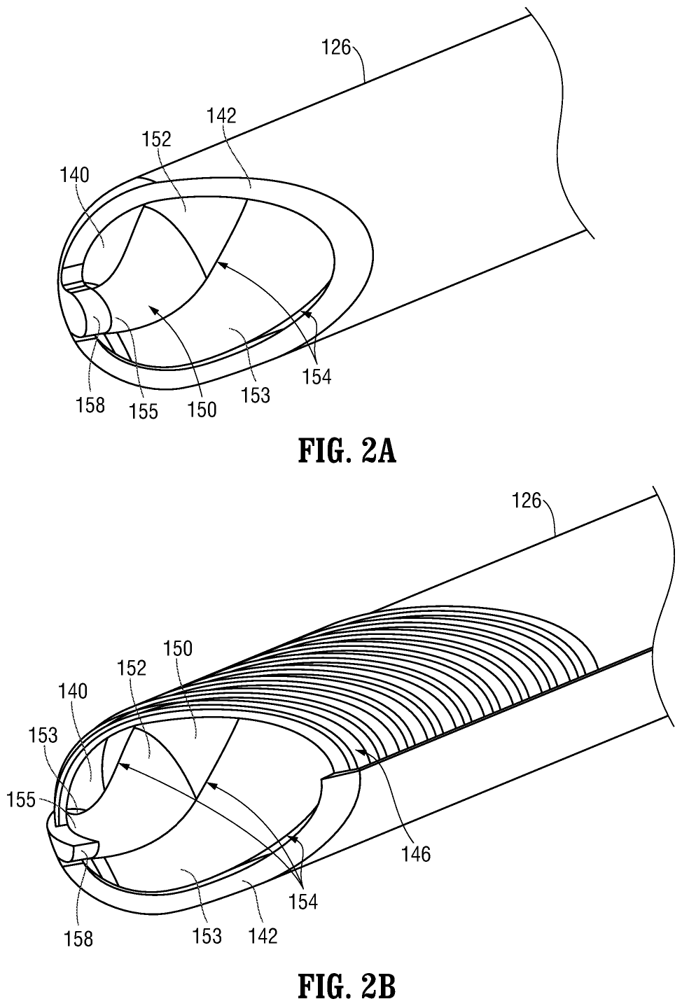

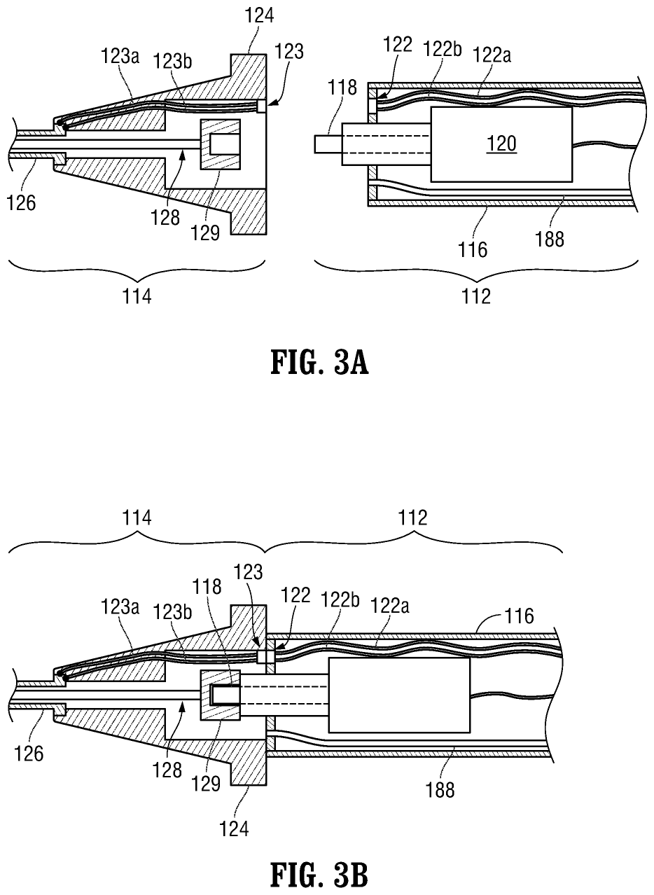

With additional reference to FIGS. 2A-3B, surgical device 110 includes a handpiece 112 that may be configured as a reusable component and an end effector assembly 114 that may be configured as a single-use, disposable component. Handpiece 112 includes a housing 116 to facilitate grasping and manipulation of surgical device 110 by a user, or facilitate mounting to a control device such as, for example, a surgical robot arm. Handpiece 112 further includes an output coupler 118 configured to operably engage end effector assembly 114, a motor 120 disposed within housing 116 and operably coupled to output coupler 118 to drive output coupler 118 and, thus, drive end effector assembly 114, and an electrical connection assembly 122 configured to electrically couple to electrical connection assembly 123 of end effector assembly 114 to enable thermal cutting-assisted tissue resection using cutting element 150, as detailed below.

Cable 170 electrically couples handpiece 112 and control console 130 with one another and, more specifically: electrically couples control console 130 with motor 120 to power and control operation of motor 120; electrically couples control console 130 with a storage device(s), e.g., a microchip(s) (not explicitly shown), associated with handpiece 112 and/or end effector assembly 114 to enable communication of, for example, identification, setting, and control information therebetween; and electrically couples end effector assembly 114 with a generator 195 disposed within control console 130 via electrical connection assemblies 122, 123 to enable selective energization of end effector assembly 114 and control of the energization thereof, as detailed below. In aspects, cable 170 is fixedly attached to handpiece 112 and releasably couplable with control console 130, although other configurations are also contemplated. As an alternative to the above-detailed configuration, motor 120 may be remotely disposed, e.g., within control console 130 and, in such aspects, cable 170 may mechanically or electromechanically couple motor 120 with handpiece 112. Further, manually-powered actuation, pneumatically-powered actuation, and/or other actuation configurations aside from an electric motor are also contemplated, whether disposed at handpiece 112 or remotely, e.g., at control console 130.

End effector assembly 114 includes a proximal hub 124 configured to releasably engage housing 116 of handpiece 112 to releasably mechanically engage end effector assembly 114 with handpiece 112. End effector assembly 114 further includes an outer shaft 126 extending distally from proximal hub 124 and an inner shaft 128 extending through outer shaft 126. A proximal end portion of inner shaft 128 extends into proximal hub 124 wherein an input coupler 129 is engaged with inner shaft 128. Input coupler 129 is configured to operably couple to output coupler 118 of handpiece 112 when proximal hub 124 is engaged with housing 116 such that, when motor 120 is activated to drive rotation of output coupler 118, input coupler 129 is driven to rotate in a corresponding manner to thereby rotate inner shaft 128 within and relative to outer shaft 126. Output and input couplers 118, 129, respectively, may be directly coupled to achieve an output to input ratio of 1:1, e.g., wherein the rotation output from output coupler 118 equals the rotation input to input coupler 129, or may be amplified or attenuated, e.g., using suitable gearing (not shown), to achieve an output to input ratio of greater than or less than 1:1. Inner shaft 128 is coaxially disposed on a longitudinal axis of outer shaft 126 and is configured to rotate about the longitudinal axis. As an alternative to or in addition to rotation of inner shaft 128 relative to outer shaft 126, inner shaft may be configured to reciprocate within and relative to outer shaft 126, e.g., via a cam-follower and helical track mechanism or other suitable mechanism operably coupled between input coupler 129 and inner shaft 128.

Outer shaft 126, as noted above, extends distally from proximal hub 124 and, in some configurations, is stationary relative to proximal hub 124, although other configurations are also contemplated. Outer shaft 126 may define a window 140 through a portion of a side and/or end wall thereof towards a distal end thereof to provide access to cutting element 150 which is rotatably disposed within outer shaft 126. At least a portion of the edge 142 of outer shaft 126 that extends about and defines window 140 may be a sharpened cutting edge and/or a dull edge. Further, in aspects, outer shaft 126 and inner shaft 128 may be flexible, e.g., steerable, malleable, pre-bent, or otherwise configured to define one or more non-linear shapes to better position the distal end portions thereof for resecting tissue.

Continuing with reference to FIGS. 1-3B, outer shaft 126 may be formed from or coated with an electromagnetically inert material, e.g., a polymeric material, or may be formed from (with or without coating) an electromagnetic material, e.g., a metal, that is inductively heated with relatively low efficiency as compared to the inductive heating efficiency of cutting element 150. Alternatively or additionally, outer shaft 126 may include shielding embedded within or disposed about an exterior surface thereof to inhibit the passage of electromagnetic energy therethrough. Outer shaft 126 includes an electromagnetic induction coil 146 embedded therein (inwardly of any shielding, if provided) or disposed thereon, e.g., on an interior surface thereof. Coil 146 may extend along a distal portion of outer shaft 126, e.g., at least partially overlapping window 140 and/or extending proximally and/or distally from window 140. In some configurations, coil 146 extends from a distal end of outer shaft 126 proximally beyond window 140 and may extend, in aspects, at least 5% of an exposed length of outer shaft 126 or at least 10% of an exposed length of outer shaft 126.

Coil 146 is configured to electrically couple to an electrical connection assembly 123 of end effector assembly 114. Electrical connection assembly 123, more specifically, may include a pair of electrical connectors 123a, 123b that are electrically isolated from one another and adapted to connect to first and second end portions of coil 146, e.g., via suitable connectors or conductors extending through outer shaft 126, embedded within outer shaft 126, formed on outer shaft 126, etc. Electrical connection assembly 123, in turn, is configured to electrically couple to electrical connection assembly 122 of handpiece 112 upon engagement of end effector assembly 114 with handpiece 112. Electrical connection assembly 122 may include a pair of electrical connectors 122a, 122b that are electrically isolated from one another and adapted to connect to electrical connectors 123a, 123b, respectively, to enable connection of different potentials of electrical energy to the first and second end portions of coil 146 to enable energization thereof. Connectors 122a, 122b may extend from handpiece 112 through cable 170 to connect to generator 195 of control console 130, thus connecting coil 146 to a source of energy.

Cutting element 150, as noted above, is rotatably disposed within outer shaft 126, and is positioned to at least partially overlap with window 140 defined through outer shaft 126. Cutting element 150 may extend the same length as coil 146, may be shorter than and within the longitudinal bounds of coil 146, or may extend beyond coil 146 in proximal and/or distal directions. Cutting element 150 may form part of, the entirety of, or may be distinct from and coupled to inner shaft 128. Regardless of the particular configuration, inner shaft 128 is rotatable within and relative to outer shaft 126 to thereby rotate cutting element 150 within and relative to outer shaft 126. Cutting element 150, as illustrated, defines a screw-shaped configuration including a body 152 having one or more helical-shaped cut-outs 153 that define one or more helical-shaped cutting edges 154. Cutting element 150 may alternatively define any other suitable configuration to facilitate tissue resection including one or more cutting edges and/or dull edges such as, for example: a corkscrew-shaped configuration; a hook-shaped configuration; a body including a plurality of barbs, projections, or other sharp or blunt cutting features; a body defining one or more cutting apertures, slots, and/or other openings, etc., combinations of the above, or any other suitable configuration. Cutting element 150 includes a distal end portion 155 at the distal end of body 152 that is rotatably received within a hub 158 disposed on an interior surface of outer shaft 126, e.g., an interior distal surface of outer shaft 126, to translationally fix and rotationally support cutting element 150 within outer shaft 126, although in other configurations distal end portion 155 of cutting element 150 is not supported by or in contact with the interior distal surface of outer shaft 126.

Cutting element 150 may be formed from a ferromagnetic material and, in aspects, a ferromagnetic material, e.g., a metal, that is inductively heated with relatively high efficiency as compared to the inductive heating efficiency of outer shaft 126 (in configurations where outer shaft 126 is electromagnetic). In aspects, cutting element 150 is formed from a ferromagnetic material such that when an electromagnetic field is applied, e.g., from coil 146, the cutting element 150 is heated up to its Curie point (thus providing automatic, Curie-point temperature control).

Referring still to FIGS. 1-3B, motor 120 of handpiece 112, as noted above, is activated to drive rotation of inner shaft 128 relative to outer shaft 126. Control console 130, coupled to motor 120 via cable 170, enables selective powering and controlling of motor 120 and, thus, selective rotation of inner shaft 128 (and cutting element 150) relative to outer shaft 126 to resect tissue extending into window 140 of outer shaft 126. Control console 130 and, more specifically, generator 195 thereof, may be configured to energize coil 146 to heat cutting element 150 (via electromagnetic inductive heating) simultaneously with the activation of rotation of inner shaft 128, in overlapping relation therewith, independently thereof, or in any other suitable manner such that, in at least some modes and/or portions of operation, energization of coil 146 and the resultant thermal heating of cutting element 150 may facilitate cutting tissue as cutting element 150 is rotated within and relative to outer shaft 126 adjacent window 140 thereof.

Outflow tubing 180 includes a distal end 184 configured to couple to handpiece 112 and a proximal end 186 configured to couple to collection vessel 160. More specifically, handpiece 112 defines an internal conduit 188 that couples distal end 184 of outflow tubing 180 with the interior of outer shaft 126 in fluid communication therewith such that fluid, cut tissue, and debris drawn into outer shaft 126 may be suctioned, under vacuum, e.g., from a vacuum pump 139 of control console 130, through end effector assembly 114, handpiece 112, and outflow tubing 180, to collection vessel 160.

Referring back to FIG. 1, collection vessel 160, as noted above, is coupled to proximal end 186 of outflow tubing 180 to receive the fluid, cut tissue, and debris suctioned through end effector assembly 114 and outflow tubing 180. Vacuum tubing 190 is coupled between collection vessel 150 and vacuum pump 139 of control console 130, such that, upon activation of vacuum pump 139, negative pressure is established through collection vessel 160, outflow tubing 180, and the interior of outer shaft 126 of end effector assembly 114 to draw tissue through window 140 of outer shaft 126 to facilitate cutting thereof, and to draw fluids, cut tissue, and debris proximally through outer shaft 126, handpiece 112 and outflow tubing 180 into collection vessel 160.

As an alternative or in addition to establishing suction through interior of outer shaft 126 via vacuum pump 139, flow can be created via a pressure differential between the interior and exterior of outer shaft 126 via by pumping fluid into the uterus (or other body cavity) to establish a positive intrauterine pressure. This may cause tissue, fluid, and/or debris to pass through window 140 and into outer shaft 126. Further, this enables tissue resection upon pressing the distal end portion of outer shaft 126 into contact with tissue, e.g., against the uterine wall or polyp, and subsequent removal of the tissue through window 140 and into outer shaft 126 such that resected tissue, e.g., diseased tissue such as cancerous cells, are removed from the uterus.

Continuing with reference to FIG. 1, control console 130 generally includes an outer housing 132, a touch-screen display 134 accessible from the exterior of outer housing 132, a cable port 136 configured to receive cable 170, a vacuum tubing port 138 configured to receive vacuum tubing 190, a vacuum pump 139 disposed within outer housing 132 and operably coupled with vacuum port 138, and a generator 195 electrically coupled with cable port 136. Outer housing 132 further houses internal electronics (not shown) of control console 130. Control console 130 may be configured to connect to a mains power supply (not shown) for powering control console 130. Further, control console 130 may be configured to receive user input, e.g., use information, setting selections, etc., via touch-screen display 134 or a peripheral input device (not shown) coupled to control console 130. Operational input, e.g., ON/OFF signals, power level settings (HI power vs. LO power), thermal cutting mode and/or temperature settings, etc., may likewise be input or selected via touch-screen display 134 or a peripheral input device (not shown) such as, for example, a footswitch (not shown), a handswitch (not shown) disposed on handpiece 112, etc.

Referring again to FIGS. 1-3B, in use, upon an activation input provided to control console 130 (or to handpiece 112 in configurations wherein handswitch controls are provided), control console 130 powers and controls motor 120 of handpiece 112 to, in turn, drive inner shaft 128 of end effector assembly 114 to rotate, thereby rotating cutting element 150. The same or a different activation input may be provided to energize coil 146, thereby heating cutting element 150. Ahead of the driving of inner shaft 128, simultaneously therewith, or delayed thereafter, vacuum pump 139 of control console 130 is activated to suction fluid, tissue, and debris through window 140, outer shaft 126, handpiece 112, outflow tubing 180, and into collection vessel 160. The suction provided from vacuum pump 139 (and/or established negative pressure, where provided) also facilitates suctioning of tissue through window 140 of outer shaft 126 such that the heated, rotating cutting element 150 can resect tissue extending through window 140 for suctioning of the resected tissue along with fluid and debris to collection vessel 160. Upon cessation of the supply of energy to coil 146 and, thus, once it is no longer desired to heat cutting element 150, suction may continue for a period whereby the flow of fluid around and about cutting element 150 serves to cool cutting element 150.

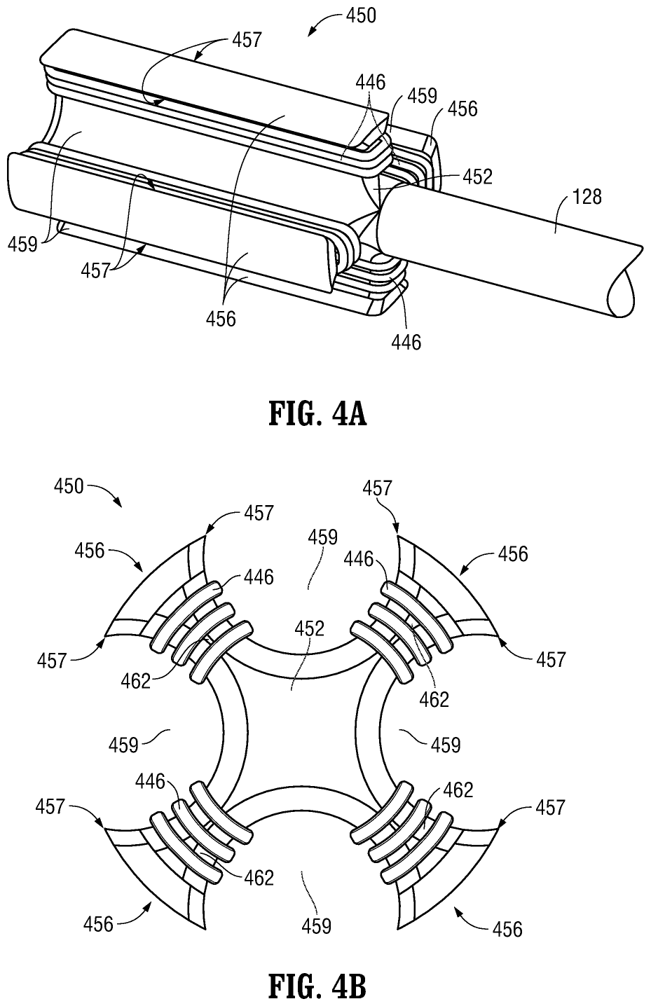

With reference to FIGS. 4A and 4B, another cutting element provided in accordance with the present disclosure is shown generally identified as cutting element 450 and engaged at a distal end portion of inner shaft 128. Cutting element 450 may be utilized within end effector assembly 114 of surgical device 110 (FIG. 1), e.g., in place of cutting element 150 (FIGS. 2A and 2B) similarly as detailed above, and may include any of the features thereof, except as specifically contradicted below. Accordingly, only differences between cutting element 450 and cutting element 150 (FIGS. 2A and 2B) and/or differences in end effector assembly 114 (FIG. 1) as a result thereof are described in detail below while similarities are summarily described or omitted entirely. Cutting element 450 may alternatively or additionally be utilized with any other suitable surgical device to facilitate cutting of tissue, as detailed below.

Cutting element 450 includes an elongated body 452 include a plurality of elongated arms 456 spaced-apart about the annular periphery of elongated body 452 and extending along at least a portion of a length thereof. Although four (4) equally-spaced arms 456 are illustrated, any other suitable arrangement in number and/or spacing may provided. The spacing of arms 456 defines an elongated recess 459 between each pair of adjacent arms 456. Each arm 456 further defines opposing longitudinal cutting edges 457 at the free end thereof such that each elongated recess 459 is surrounded on either side thereof via an elongated cutting edge 457. Arms 456 may taper in thickness from the free ends thereof inwardly towards elongated body 452 to define a neck 462 between the free end of each arm 456 and elongated body 452. Cutting element 450 may be formed from similar materials and/or in a similar manner as detailed above with respect to cutting element 150 (FIGS. 2A and 2B), or may define any other suitable configuration.

Continuing with reference to FIGS. 4A and 4B, an elongated electromagnetic induction coil 446 is disposed about the neck 462 of each arm 456. With coils 446 disposed directly about cutting element 450, a coil need not be provided on or within outer shaft 126 (FIG. 2A) as with cutting element 150 (see FIG. 2A). Electromagnetic induction coils 446 may be independently or collectively electrically coupled to a source of energy, e.g., generator 195 similarly as detailed above with respect to system 100 (see FIG. 1). More specifically, electrical connection assembly 123 (FIGS. 3A and 3B) may be configured to supply energy to each coil 446 via two or more connectors extending through, embedded within, disposed on, or otherwise coupled to inner shaft 128. Coils 446 may be formed form similar materials and/or in a similar manner as coil 146 (FIG. 2A), although other configurations are also contemplated.

In use, similarly as detailed above with respect to cutting element 150 (FIGS. 2A-2B), cutting element 450 may be selectively heated via coils 446 such that, together with rotation of cutting element 450 within and relative to outer shaft 126 (FIG. 1), tissue resection and removal is facilitated. Further, cutting element 450 and/or cutting element 150 (FIGS. 2A-2B) may also provide spot cauterization and/or coagulation adjacent the distal end portion of outer shaft 126 (FIG. 1) during use, thus helping to minimize bleeding at the area of tissue from which tissue was resected.

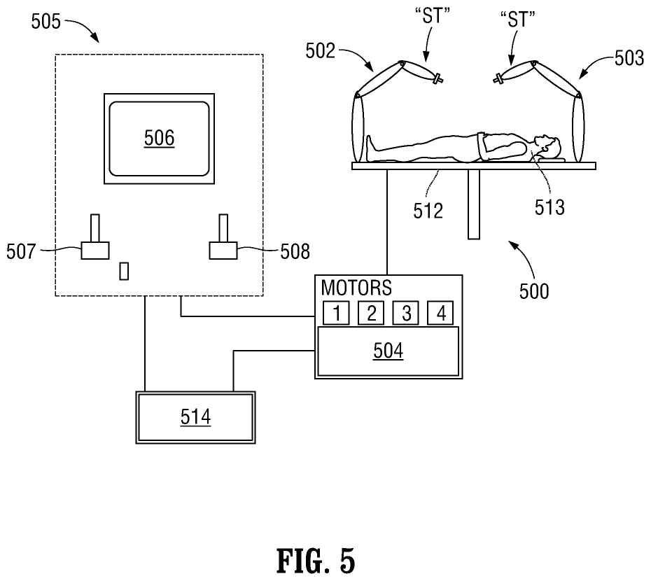

Turning to FIG. 5, robotic surgical system 500 is configured for use in accordance with the present disclosure. Aspects and features of robotic surgical system 500 not germane to the understanding of the present disclosure are omitted to avoid obscuring the aspects and features of the present disclosure in unnecessary detail.

Robotic surgical system 500 generally includes a plurality of robot arms 502, 503; a control device 504; and an operating console 505 coupled with control device 504. Operating console 505 may include a display device 506, which may be set up in particular to display three-dimensional images; and manual input devices 507, 508, by means of which a person, e.g., a surgeon, may be able to telemanipulate robot arms 502, 503 in a first operating mode. Robotic surgical system 500 may be configured for use on a patient 513 lying on a patient table 512 to be treated in a minimally invasive manner. Robotic surgical system 500 may further include a database 514, in particular coupled to control device 504, in which are stored, for example, pre-operative data from patient 513 and/or anatomical atlases.

Each of the robot arms 502, 503 may include a plurality of members, which are connected through joints, and a mounted device which may be, for example, a surgical tool “ST.” One or more of the surgical tools “ST” may be end effector assembly 114 of surgical device 110 (FIG. 1), thus providing such functionality on a robotic surgical system 500. In such a configuration, the corresponding robot arm 502, 503 may incorporate (or attach to a device incorporating) the operably features of handpiece 112 (FIG. 1) and/or be configured to couple to the other components of surgical system 100 (FIG. 1) to enable the system to operate similarly as surgical system 100 (FIG. 1), except in a robotic configuration.

Robot arms 502, 503 may be driven by electric drives, e.g., motors, connected to control device 504. Control device 504, e.g., a computer, may be configured to activate the motors, in particular by means of a computer program, in such a way that robot arms 502, 503, and, thus, their mounted surgical tools “ST” execute a desired movement and/or function according to a corresponding input from manual input devices 507, 508, respectively. Control device 504 may also be configured in such a way that it regulates the movement of robot arms 502, 503 and/or of the motors.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular aspects. Those skilled in the art will envision other modifications within the scope and spirit of the claims appended hereto.

What is claimed is:

1. A tissue resection device, comprising:
a handpiece; and
an end effector assembly extending distally from the handpiece, the end effector assembly including:
an outer shaft including a window defined therethrough towards a distal end thereof;
an inner shaft rotationally disposed within the outer shaft, the inner shaft operably coupled to the handpiece such that the handpiece is capable of rotationally driving the inner shaft relative to the outer shaft;
a cutting element disposed at a distal end portion of the inner shaft within the outer shaft and at least partially overlapping the window, the cutting element configured such that rotation of the inner shaft rotates the cutting element, the cutting element formed at least partially from a ferromagnetic material capable of being inductively heated; and
at least one electromagnetic induction coil surrounding at least a portion of the cutting element, the at least one electromagnetic induction coil adapted to connect to a source of energy to produce an electromagnetic field within the at least one electromagnetic induction coil to thereby inductively heat the cutting element.

2. The tissue resection device according to claim 1, wherein the cutting element defines at least one cutting edge configured to cut tissue upon at least one of heating of the cutting element or rotation of the cutting element.

3. The tissue resection device according to claim 1, wherein the cutting element defines a screw-shaped configuration including at least one helical cutting edge.

4. The tissue resection device according to claim 1, wherein the cutting element includes an elongated body and a plurality of elongated arms annularly spaced about the elongated body, each elongated arm of the plurality of elongated arms defining at least one elongated cutting edge.

5. The tissue resection device according to claim 4, wherein each elongated arm of the plurality of elongated arms defines an elongated cutting edge along each side thereof.

6. The tissue resection device according to claim 1, wherein the at least one electromagnetic induction coil includes an electromagnetic induction coil disposed on or embedded within the outer shaft.

7. The tissue resection device according to claim 1, wherein the at least one electromagnetic induction coil includes a plurality of electromagnetic induction coils disposed about different portions of the cutting element.

8. The tissue resection device according to claim 7, wherein the different portions of the cutting element includes arms extending from a body of the cutting element.

9. The tissue resection device according to claim 1, wherein the handpiece includes a motor and an output coupler connected to the motor, wherein the end effector assembly includes an input coupler connected to the inner shaft, and wherein the input coupler is configured to engage the output coupler to enable the motor to drive rotation of the inner shaft.

10. The tissue resection device according to claim 9, wherein the end effector assembly is configured to releasably engage the handpiece to thereby releasably couple the output and input couplers with one another.

11. The tissue resection device according to claim 1, wherein the handpiece is adapted to connect to a vacuum source to establish suction through the outer shaft, thereby facilitating drawing tissue through the window and into the outer shaft for cutting with the cutting element.

12. The tissue resection device according to claim 1, wherein the cutting element is formed from a ferromagnetic material.

13. A tissue resection device, comprising:
a handpiece; and
an end effector assembly extending distally from the handpiece, the end effector assembly including:
an outer shaft including a window defined therethrough towards a distal end thereof;
an electromagnetic induction coil disposed on or embedded within the outer shaft, the electromagnetic induction coil adapted to connect to a source of energy to produce an electromagnetic field within the outer shaft;
an inner shaft rotationally disposed within the outer shaft, the inner shaft operably coupled to the handpiece such that the handpiece is capable of rotationally driving the inner shaft relative to the outer shaft; and
a cutting element disposed at a distal end portion of the inner shaft within the outer shaft and at least partially overlapping the window and the electromagnetic induction coil, the cutting element configured such that rotation of the inner shaft rotates the cutting element, the cutting element formed at least partially from a ferromagnetic material capable of being inductively heated by the electromagnetic field produced by the electromagnetic induction coil.

14. The tissue resection device according to claim 13, wherein the cutting element defines a screw-shaped configuration including at least one helical cutting edge.

15. The tissue resection device according to claim 13, wherein the handpiece includes a motor and an output coupler connected to the motor, wherein the end effector assembly includes an input coupler connected to the inner shaft, and wherein the input coupler is configured to engage the output coupler to enable the motor to drive rotation of the inner shaft.

16. The tissue resection device according to claim 13, wherein the handpiece is adapted to connect to a vacuum source to establish suction through the outer shaft, thereby facilitating drawing tissue through the window and into the outer shaft for cutting with the cutting element.

17. A tissue resection device, comprising:
a handpiece; and
an end effector assembly extending distally from the handpiece, the end effector assembly including:
an outer shaft including a window defined therethrough towards a distal end thereof;
an inner shaft rotationally disposed within the outer shaft, the inner shaft operably coupled to the handpiece such that the handpiece is capable of rotationally driving the inner shaft relative to the outer shaft;
a cutting element disposed at a distal end portion of the inner shaft within the outer shaft and at least partially overlapping the window, the cutting element configured such that rotation of the inner shaft rotates the cutting element, the cutting element formed at least partially from a ferromagnetic material capable of being inductively heated and including a body having a plurality of arms disposed annularly thereabout; and
a plurality of electromagnetic induction coils, each electromagnetic induction coil of the plurality of electromagnetic induction coils disposed about an arm of the plurality of arms, each electromagnetic induction coil adapted to connect to a source of energy to produce an electromagnetic field therewithin to thereby inductively heat the corresponding arm of the cutting element.

18. The tissue resection device according to claim 17, wherein each arm of the plurality of arms defines an elongated cutting edge along each side thereof.

19. The tissue resection device according to claim 17, wherein the handpiece includes a motor and an output coupler connected to the motor, wherein the end effector assembly includes an input coupler connected to the inner shaft, and wherein the input coupler is configured to engage the output coupler to enable the motor to drive rotation of the inner shaft.

20. The tissue resection device according to claim 17, wherein the handpiece is adapted to connect to a vacuum source to establish suction through the outer shaft, thereby facilitating drawing tissue through the window and into the outer shaft for cutting with the cutting element.
